(19) United States
(12) Patent Application Publication
(10) Pub. No.: US 2007/0166714 A1
(43) Pub. Date: Jul. 19, 2007

(54) Methods for preparing gene chips and use thereof

(75) Inventors: Pierre Lindenbaum, Versailles (FR); Philippe Gesnouin, Saint-Fargeau-Ponthierry (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: INTEGRAGEN, EVRY FRANCE (FR)

(21) Appl. No.: 10/561,575

(22) PCT Filed: Jun. 18, 2004
(86) PCT No.: PCT/FR04/01533

§ 371 Date: Dec. 19, 2005

(30) Foreign Application Priority Data
Jun. 20, 2003 (FR) 03/07508

(51) Int. Cl. C12Q 1/68; C12M 1/34; H01L 21/00

(52) U.S. Cl. 435/6; 435/287.2; 438/1; 257/E51.02

(57) ABSTRACT

The invention relates to methods and compositions for producing biochips as well as to the use of these biochips in diverse fields, from functional genomics to diagnosis, for example, particularly in research or in the medical field. The invention also relates to tools and methods for selecting polynucleotides that permit the production of improved biochips. The inventive biochips contain, in particular, BAC clones.

This application relates to methods and compositions for producing or manufacturing biochips, as well as to the use of these biochips in diverse fields, from functional genomics to diagnosis, for example, particularly in research or in the medical field. It also relates to tools and methods for selecting polynucleotides that permit the production of improved biochips.

With the development of genomics and miniaturisation techniques, new strategies for identifying genes, for analysis or diagnosis have come to light. These strategies are based in particular upon hybridisation reactions between a test sample, the content of which one wishes to analyse, and a library of polynucleotides which are immobilised on a support. These types of approach are or will be used in diagnosis, research, pharmacogenomics, etc., in order to analyse a population of nucleic acids or a biological sample.

Different types of polynucleotide can thus be immobilised on supports, according to the type of application required. Thus, oligonucleotides, PCR fragments, BACs, genes or fragments of particular genes, RNAs, etc., have been immobilised on supports. Furthermore, these can be polynucleotides with a pre-defined or known sequence, or with a random sequence, or a combination. Different strategies for producing these supports have also been introduced, which are classified in two main categories: in situ synthesis or coupling. In the in situ synthesis methods, the polynucleotides are synthesised by direct elongation on the support, for example by photolitography (see for example patent U.S. Pat. No. 5,510,270). This technique is essentially limited to the synthesis of oligonucleotide biochips. In the coupling techniques, the polynucleotides are immobilised by depositing them on the support, after synthesis and/or selection.

Different techniques have been described, including direct coupling on the support, or an interaction with a complementary oligonucleotide, or coupling by means of a spacer arm, etc. Preparation by coupling makes it possible to widen the scope of biochips to any type of polynucleotide, as indicated above. Moreover, different types of support have also been proposed, such as supports made of (or with a base of) glass, plastic, polymer, metal, biological materials, silicons, nylon, etc.

Within the framework of this application, the generic term “biochip” will be used to refer to any support on which polynucleotides are immobilised. Polynucleotides are generally immobilised on the surface of the support or on an area of the same, so as to be accessible for a hybridisation reaction. The immobilisation can be covalent or not, ordered or not, dense or not, etc. Preferably, it is covalent and ordered.

There are various applications for biochips, ranging from research to diagnosis. Thus, chips are used for researching differences in the expression of genes, genetic alterations, for comparing samples, for locating markers, in sequencing, for comparing numbers of copies of genes, etc.

For these different applications, a sample to be analysed is put in contact with the biochip and a hybridisation signal is detected. In general, the nucleic acids of the sample are marked in advance, so as to facilitate detection. According to the amplitude of the signal detected, the position of the signal, etc., it is possible to determine the presence, in the sample, of a particular nucleic acid, of an altered form of a gene or messenger, a level of expression, etc. Numerous approaches have been proposed for the marking of samples, the putting in contact of the samples and of the chip, the reading of hybridisation signals, the analysis of results, etc.

However, there is currently a need for improved methods for producing biochips, the composition and the structure of which are better controlled, and which make possible more reliable applications and readings.

### SUMMARY OF THE INVENTION

This application now describes methods and tools for the production of particular biochips. It also describes the use of these biochips in diverse fields, from functional genomics to diagnosis for example, particularly in research or in the medical field. It also relates to tools and methods for selecting polynucleotides that permit the production of improved biochips. The biochips according to the invention are characterised in particular by the fact that they comprise a plurality of polynucleotides forming a set (or a collection) representative of the genome of an organism being considered (e.g., of its sequence, of its organisation, etc.). The genome being considered is preferably a human genome. Such biochips are particularly adapted to locate, position or map any nucleic acid of interest, or for diagnostic applications or in pharmacogenomics, to evaluate the presence, or the levels of expression (absolute or relative) of genes, in subjects or in cells in culture, for example.

A first objective of the invention is more particularly a method for producing a biochip including a support on which a set of polynucleotides is immobilised, characterised in that it includes:
(ii) selecting, from a plurality of BAC clones including a nucleic insert corresponding to or specific to a portion of a genome, preferably a human genome, a set of BAC clones including a single insert in said genome, the BAC clones of the selected set including nucleic inserts distributed substantially uniformly over the genome and, preferably, spaced apart from one another by an interval of an order of about 1 Mb, and
(iii) depositing, on a support, the clones selected in this way, or the nucleic inserts that they contain, or part of them, in conditions enabling the deposited clones or inserts to hybridise with a nucleic acid having a complementary sequence.

A more particular objective of the invention is notably a method for producing a biochip including a support on which a set of marker polynucleotides of a genome, in particular of the human genome, is immobilised, characterised in that it includes
(i) obtaining BAC clones including a nucleic insert corresponding to or specific to a portion of a genome, preferably human,
(ii) selecting, from the BAC clones obtained in this way, a set of BAC clones including a single insert in said genome, the BAC clones of the selected set including nucleic inserts distributed substantially uniformly over the genome and spaced apart from one another by an interval of an order of about 1 Mb, and
(iii) depositing, on a support, the clones selected in this way, or the nucleic inserts that they contain, or part of them, in conditions enabling the deposited clones or inserts to hybridise with a nucleic acid having a complementary sequence.

Another aspect of the invention relates to a biochip, characterised in that it includes a support on which a set of BAC clones is immobilised including a nucleic insert corresponding to or specific to a portion of a genome, preferably human, each clone including a single insert in said genome, the BAC clones of the set including nucleic inserts distributed substantially uniformly over said genome and advantageously spaced apart from one another by a regular interval of an order of about 1 Mb. Preferably, the BAC clones are arranged in a specified way on the support. In a preferred variation, the support is a glass slide.

Another aspect of the invention is the use of a biochip such as defined above for genetic analysis, in particular for the identification of genes, for genetic mapping, for diagnosis, in pharmacogenomics, etc.

Another aspect of the invention relates to a method for identifying or locating a nucleic acid on the human genome, including placing a test nucleic acid in contact with a biochip as defined above in conditions enabling hybridisation between complementary sequences, detection of a hybridisation signal, and identification of the position of the nucleic acid on the genome by identifying the clones involved in the hybridisations.

Another aspect of the invention relates to a method for detecting the presence or the abundance (e.g., absolute or relative levels of expression) of a gene in a biological sample, including putting the sample in contact with a biochip as defined above in conditions enabling hybridisation between complementary sequences, and detection of a hybridisation signal, said signal being indicative of the presence or of the abundance of a gene in the sample. Advantageously, the biological sample is of human origin and includes nucleic acids (biopsy, cell culture, cell lysate, biological fluid, tissue, organ, etc.). The sample can be treated in advance, so as to make accessible (or to favour access to) nucleic acids for a hybridisation reaction.

The invention thus provides new methods and tools for producing improved biochips, comprising a plurality of BAC clones forming a set (or a collection) which is representative of the human genome (of its sequence, of its organisation, etc.). This application describes methods for selecting, preparing, depositing (e.g., “spotting”) on the support and hybridisation of the supports obtained in this way with biological samples, making it possible to map, locate and identify genes of interest.

### DETAILED DESCRIPTION OF THE INVENTION

The term BAC clone or “Bacterial Artificial Chromosome” indicates a bacterial clone comprising a nucleic insert corresponding to or specific to a portion of a genome, preferably a human genome. The term BAC clone indicates either the bacterial clone comprising the nucleic insert, or a vector extracted (or isolated) from the bacterial clone, and including the nucleic insert, either the nucleic insert itself, or a part of the same, or else the total nucleic acids of the bacterium. The BACs are vectors adapted to cloning fragments of DNA of considerable length, and are used to build libraries. According to the data published, it is known that several thousand different BAC clones currently exist which are available in collections, each comprising a distinct nucleic insert representative of a segment of human genome.

In order to implement this invention, the BAC clones can be obtained, collected or gathered from numerous sources, such as data bases, sequencing data, collections of samples, etc. Sequencing of the human genome has made it possible to reveal and make available numerous makers or clones, corresponding to regions of the human genome. These markers or clones now cover the whole sequence of the human genome, but are not in order or classified sufficiently completely or precisely so as to be able to be used effectively. Thus, these multiple clones comprise overlapping, redundant, non-specific, mis-located, sometimes non-characterised etc. clones. Due to their plurality, complexity and diversity, it has not been possible to exploit these clones satisfactorily until now for the production of validated diagnostic or analytic products. This application now proposes producing biochips from BAC clones. It advantageously proposes new tools and methods making it possible to select validated sets of clones.

In the methods of the invention, BAC clones are obtained so as to supply a collection of clones able to cover the whole sequence of the genome being considered, preferably a human genome. Public sources of BAC clones are in particular BACPAC resources (chori.org), Research Genetics (resgen.com) or the Sanger centre (sanger.ac.uk, CloneRequest). These collections are accessible to the public, for example on the internet, and are well known to experts in the field.

In these collections, clones are generally presented in the form of bacteria clones including a BAC vector containing the nucleic insert. BAC clones obtained from these sources are therefore preferably in the form of bacterial culture, which can be stored, analysed, replicated, etc. The BAC vector carrying the insert can also be isolated and analysed, amplified, sub-cloned, etc. The clones obtained in this way are typically stored in culture boxes, or in any appropriate container (tube, vial, flask, etc.). They can be lyophilised, frozen, etc.

Preferably, sufficient BAC clones are gathered so as to obtain a collection able to cover the whole sequence of the genome being considered, preferably a human genome. BAC clones for which certain structural and/or functional information is available (e.g., in situ hybridisation data (“FISH”, partial sequence, etc.) are preferred. The selection of BAC clones is then a very important feature of the invention. Indeed, it makes it possible to supply, from numerous clones, a set of validated clones, which is coherent and usable for the production of biochips adapted to reliable mapping or identification experiments.

The selection is advantageously made by elimination, should the occasion arise, according to several successive cycles during which the selection is more and more profound and the quality of the clones increased.

The selection of the BAC clones of interest can advantageously be made by means of a computer programme, or using, in certain steps, computerised decision rules. In particular, the invention is adapted to the production of chips for analysis of the human genome.

In a preferred embodiment, the selection of clones includes:
(a) elimination of non-single clones;
(b) elimination of clones sharing a same STS;
(c) elimination of STS which are marked at at least two different places on the genome being considered, in particular human;
(d) classification of the clones as a function of their position on the genome, thus defining neighbouring clones; and
(e) additional elimination of clones, by applying an iterative method, so as to obtain in particular a substantially uniform distribution over the genome being considered, in particular human, of the clones finally selected.

The order of steps (a) to (e) can be interchanged. Furthermore, certain of these steps can be implemented simultaneously. It will be noted however that step (e) can not be implemented before step (d).

Advantageously, selection steps (a) to (e) or part of them are repeated (one or more selection cycles can thus be implemented), until a set of clones as defined above is obtained. Typically, two clones are first of all analysed, then additional clones are progressively introduced to the analysis, until most of, and preferably the whole genome, is scanned.

Step (a) therefore includes elimination of the non-single clones, i.e. clones which are marked at at least two different places on the genome being considered, preferably human. The term “marked” indicates that the nucleic insert that these clones contain is present in several positions in a genome or specific (or complementary) to several regions in the genome. In so far as these clones can hybridise with distinct regions of the genome being considered, they can not make it possible to effectively locate a fragment of nucleic acid and so are advantageously eliminated.

The non-uniqueness of a clone can be demonstrated in different ways, such as for example by compiling or analysing information known for a clone (position, marker, sequence, etc.), by computer analysis of the sequence of the nucleic insert that it contains (if its sequence is made up from repeated or consensus motifs, it will not a priori be unique in character), by biological experiments (e.g., in situ hybridisation, etc.).

Step (b) includes elimination of the clones sharing a same STS “Sequence Tagged Site”. The STS is the site on the genome “tagged” by a sequence, i.e., in this case, the target site of a BAC clone on the human genome. When several clones share a same STS, these clones are redundant and make the biochip more complex. This application therefore proposes a selection of clones including elimination of the clones sharing a same STS increasing the specificity of the biochip. The STS of a clone can be identified by techniques known in their own right, such as for example using information available for each of the clones, regarding the sequence of the nucleic insert, in situ hybridisation data, etc.

The STS of the clones are then compared and when two clones share a same STS, one of them is eliminated.

Step (c) includes elimination of the STS which are marked at at least two different places on the genome being considered.

Step (d) includes classification of the BAC clones as a function of their position on the genome. This can be implemented by analysing the known marks, or by any other method known in its own right by experts in the field. This step leads to the definition of “neighbouring clones”, i.e. immediately adjacent clones on the genome being considered.

In order to implement this classification, one marks the position of the nucleic insert of each BAC clone on the genome being considered, for example by representing the latter in the form of a scale graduated from 0 to 100. Each nucleic insert can then be represented by a segment of the genome, of co-ordinates xᵢ and yᵢ, with yᵢ>xᵢ, on the scale graduated from 0 to 100.

The BAC clones noted as rᵢ can thus be classified on this graduated scale in ascending order of their co-ordinates xᵢ, or else, as a variation, in ascending order of their co-ordinates yᵢ. One thus obtains the following classification, in the case where one compares the co-ordinates xᵢ:
0<xᵢ, < . . . <xᵢ< . . . <xₙ<100.

Step (e) is a step for eliminating BAC clones, from all of the clones previously classified {rₗ, . . . rₙ}, in order to obtain a set E of finally selected clones. This step starts with a first sub-step (eₗ) of extracting from a sub-set E′ BAC clones likely to be eliminated from the E set of finally selected clones, this sub-step of extraction being implemented by applying a first rejection criterion. This first rejection criterion is based upon the calculation of an algebraic variance, in the following referred to as the “variance” between BAC clones. The variance between two BAC rᵢ (xᵢ, yᵢ) and rⱼ (xⱼ, yⱼ) clones, with j>i, is defined by the following relation:
d(rᵢ, rⱼ)=xⱼ−yᵢ.

It will be noted that the variance between two BAC clones takes a negative value if the two BAC clones overlap, a zero value if the second BAC clone is located in the extension of the first, and a positive value if these two BAC clones do not have a common part.

It is important that the finally selected BAC clones of the set E include nucleic inserts distributed substantially uniformly over the human genome, i.e. that there is not too great a variance between two successive neighbouring BAC clones selected. In order to do this, the first rejection criterion is defined as a threshold S the value of which corresponds to the maximum variance tolerated between two neighbouring BAC clones in the E set of the finally selected clones. For example, S=1.5 Mb, and this translates by a variance value s on the scale graduated from 0 to 100 of the human genome.

For every i between 2 and n, in order to determine whether a BAC rᵢ clone must belong to the sub-set E′ of clones likely to be eliminated, the variance is calculated between the clone rᵢ₋₁ and the clone rᵢ₊₁. For the BAC r₁, clone the variance d(0, r₂)=x₂ is calculated. Finally, for the rₙ clone the variance d(rₙ₋₁, 100)=100 −Yₙ₋₁ is calculated. If the variance calculated is less than the value s, the corresponding rᵢ clone belongs to E′.

During a second sub-step (e₂), a second criterion is applied to the elements of the sub-set E′ of clones likely to be eliminated, so as to determine the single clone of this sub-set which will effectively be eliminated.

In order to do this, each BAC rᵢ clone is associated with a list of properties from which is calculated for example a cost function f(rᵢ) for each BAC clone. This makes it possible to select in the sub-set E′, the clone which maximises this cost function. This clone is then eliminated. In a classic manner, the cost function has positive values and is even higher than the corresponding clone is likely to be eliminated.

As a variation, one can replace the calculation of a cost function by a system based on rules which make it possible to compare the lists of properties of two clones and to take a decision to eliminate one of the two clones being compared.

The list of properties attached to a BAC clone can for example comprise an availability parameter, a parameter defining the original collection of the BAC clone, a parameter relating to validation by in-situ hybridisation.

One can also imagine other properties such as a parameter linked to the covering of one BAC clone with another, making it possible preferably to eliminate the BAC clones covering one another, or a parameter encouraging elimination preferably of BAC clones which generate variances between the previous clone and the following clone above a threshold S′, chosen as a function of the threshold S, such that the selected nucleic inserts are spaced apart from one another by a more or less regular interval. By choosing a value S=1.5 Mb and S′=0.7 Mb, one obtains a set of selected BAC clones the nucleic inserts of which are spaced apart from one another by intervals of an order of about 1 Mb, on the human genome. In the same way as for S, the value S′ translates by a variance value s′ on the scale graduated from 0 to 100 of the human genome.

The succession of the two sub-steps (e₁) and (e₂) described above is repeated until one can no longer eliminate a BAC clone, without creating a variance greater than the threshold S on the human genome, i.e. when the sub-set E′ of BAC clones likely to be eliminated obtained during step (e₁) is the empty set.

The first sub-step (e₁) of extracting from the sub-set E′ and the second sub-step (e₂) of eliminating a clone from this sub-set E′ can also be articulated in the following way (taking as an example the case of calculating a cost function):
step 1—initialisation of an index k at the value of zero and an index F at the value of zero;
step 2—for every i ranging from 1 to n:
   (e1) it is determined whether rᵢ belongs to the sub-set E′;
   (e2) if “yes”, it is tested whether f(rᵢ) is greater or equal to F and if this is the case, k is given the value i and F the value f(rᵢ);
step 3—if k is zero, this means that the sub-set E′ is the empty set and in this case, one passes on to step 6;
step 4—otherwise clone rₖ is eliminated and the remaining clones are re-ordered by decrementing n by one unit;
step 5—one passes on to step 1;
step 6—end of the process.

As indicated above, the order of steps (a) to (e) can be modified, in particular the order of steps (a) to (c). Furthermore certain steps can be implemented simultaneously. The selection is advantageously made by using a particular computer programme able to analyse and compare the data for each BAC clone. With regard to this, this application also proposes new tools which facilitate implementation of steps (a) to (c) starting with a very high number of initial BAC clones, in particular computer tools.

Thus, this invention describes the CloneTrek tool, which is an application suite, the purpose of which is to select sub-sets of objects (BAC clones) as a function of their properties (validation by in situ hybridisation, original collection, availability) and of their location on an axis 1-D (the human genome). Moreover, CloneTrek offers the possibility of graphically representing the maps obtained, of providing a map with additional data, of comparing maps, of generating input files for certain types of robots for sub-culturing plaques, calculating statistics on the BACs of a collection, etc.

All of the CloneTrek programmes use and exchange data formatted in XML, (“eXtended Markup Language”) according to proprietary (XMLMap and XMLPlateHandler) DTD (“Document Type Definition”). Programmes for importing data from internal and public resources have been developed so as to translate these data in these formats.

The clone tag algorithm and the data used are described below:
Objective: to select BACs (bacterial clones including a given human insert) so as to use them as a position tag on a DNA chip.
Initial data:
   STS_aliases: list of referenced STS, associated data including FISH, supplied by NCBI;
   BAC_Clones: list of referenced BACs, associated data (including FISH) collected (NCBI, etc.).

Position: files supplied by NCBI and Golden Path listing by position on the genome the information which is attached, in particular the clones and the STS. For these latter versions, we essentially use the NCBI data.

Suppliers' collection: the BAC clones must be ordered from a supplier who has them available in the form of collections.

Even if the biochips preferred by the invention make it possible to cover the whole of a human genome, it is also possible to produce biochips covering just a portion of a genome (for example one or more chromosomes).

The method described above comprising steps (a) to (e) includes, in this case, the following steps:
first of all the clones and the STS are eliminated which are marked at two different places on the genome position being considered, in accordance with steps (a) to (c) described above;
then, in accordance with steps (d) and (e), for each chromosome:
   the clones are ordered on the chromosome, as a function of their position or of the position of the STS to which they refer; and
   additional elimination of clones, by applying an iterative method, so as to obtain in particular a substantially uniform distribution over the human genome of the finally selected clones.

Following selection of the BAC clones, the latter (or the nucleic inserts that they contain, or part of these clones or inserts), are deposited on a support, in conditions enabling the deposited clones or inserts to hybridise with a nucleic acid having a complementary sequence. Different techniques can be used for depositing the clones or inserts, such as direct coupling on the support, or an interaction with a complementary oligonucleotide, or coupling by means of a spacer arm, of bi-functional agents, etc. In general, so as to enable the deposited clones or inserts to hybridise with a nucleic acid having a complementary sequence, it is preferable for the clones or inserts to be linked to the support by one of their ends. Different methods are possible, such as the use of a spacer molecule (WO99/51773), of a support coated with functional groups such as polyethyleneimmine (GB2,197,720) or avidine (WO97/18226), or of arborescent arms (EP 647 719, WO99/61662, WO99/10362). Other immobilisation techniques are described for example in patents U.S. Pat. No. 4,925,785 and EP 373 203. Moreover, different types of support can be used, such as supports which are level or not, rigid or not, based upon different materials such as glass, plastic, polymer, metal, biological materials, silicons, nylon, etc. As an illustration, one can cite nylon membranes, glass slides, silicon plates, etc.

In a preferred embodiment, the support is a glass slide. Depositing onto the glass slide can be implemented by depositing samples directly onto the slide, or after pre-treatment of the slide so as to encourage interaction with nucleic acids. Slides which can be used are for example glass slides covered with amino-silane (for example GAPS II slides, Corning).

In a particularly preferred way, depositing is achieved in an ordered fashion, i.e. according to a (pre-)determined arrangement and/or density.

Advantageously, each clone or insert is positioned on an identifiable zone (a cell) of the support. Depositing can be implemented advantageously by means of a robot. The density of the clones or inserts on the support can vary, as a function of the number of distinct clones or inserts and of the surface of the support. In general, less than 1000 distinct clones or inserts are deposited on a surface of 1 cm². Of course, each clone is generally present in several copies, so as to increase the sensitivity of the biochip.

Before being deposited on the support, the clones of the selected set can be sub-cultured, amplified, characterised, stored, etc. In this way, it is possible and easy to reproduce biochips of the invention. With regard to this, an alternative embodiment of the invention is a method for producing a biochip including a support on which is immobilised a set of marker polynucleotides of the human genome, characterised in that it includes:
(ii) selecting, from a plurality of BAC clones including a nucleic insert corresponding to or specific to a portion of a human genome, a set of BAC clones including a single insert in the human genome, the BAC clones of the selected set including nucleic inserts substantially uniformly distributed over the human genome and spaced apart from one another by an interval of an order of about 1 Mb,
(iii) amplifying the BAC clones of the selected set and/or the nucleic inserts that they contain, and
(iv) depositing in an ordered fashion, on a support, the clones selected in this way, or the nucleic inserts that they contain, or part of them, in conditions enabling the deposited clones or inserts to hybridise with a nucleic acid having a complementary sequence.

A more specific objective of the invention is a method for producing a biochip including a support on which is immobilised a set of polynucleotides, characterised in that it includes
(i) obtaining BAC clones including a nucleic insert corresponding to or specific to a portion of a human genome,
(ii) selecting, from the BAC clones obtained in this way, a set of BAC clones including a single insert in the human genome, the BAC clones of the selected set including nucleic inserts distributed substantially uniformly over the human genome and spaced apart from one another by an interval of an order of about 1 Mb,
(iii) amplifying the BAC clones of the selected set and/or of the nucleic inserts that they contain, and
(iv) depositing, in an ordered fashion on a support, the clones selected in this way, or the nucleic inserts that they contain, or part of them, in conditions enabling the deposited clones or inserts to hybridise with a nucleic acid having a complementary sequence.

Another aspect of the invention relates to a biochip, characterised in that it includes a support on which is immobilised a set of BAC clones including a nucleic insert corresponding to or specific to a portion of a human genome, each clone including a single insert in the human genome and carrying a STS which is not shared by another insert of the BAC clones of the set, the BAC clones of the set including nucleic inserts distributed substantially uniformly over the human genome and spaced apart from one another by a regular interval of an order of about 1 Mb. Preferably, the BAC clones are arranged in a specified way on the support. In a preferred variation, the support is a glass slide. Of course the biochip of the invention can furthermore include other polynucleotides, which can be BAC clones or not. Thus, the biochip can include control polynucleotides; of various origin, nature and size.

Another objective of the invention is the use of a biochip as defined above for identifying genes, for genetic mapping, for diagnosis, in pharmacogenomics, etc. The biochips of the invention can be used in research, for identifying genes, cloning, the analysis of differences in expression between cells or tissues, etc. They can also be used in diagnosis or pharmacogenomics methods, in order to detect genomic or genetic alterations, in order to detect differences in the expression of genes, etc.

Particular Applications are Notably:

detecting the change in copy number of a chromosome or of a portion of a chromosome associated with a disease,
toxicological studies: identifying toxic compounds likely to induce modifications to the copy number of a portion of the genome (toxicogenomics),
detecting translocation
characterisation of hybrid panels from irradiation for different species,
the non-quantitative study of the expression of genes, in particular those which are not present in the so-called expression chips,
the scale study of the whole genome of the DNA protein interactions,
any application based on genomic mapping and/or change in ratio,
etc.
   In this regard, a particular objective of the invention is a method for identifying or locating a nucleic acid on the human genome, including placing a nucleic acid in contact with a biochip as defined above in conditions enabling hybridisation between complementary sequences, detection of a hybridisation signal, and identification of the position of the nucleic acid on the genome by identifying the clones involved in the hybridisations.

The nucleic acid tested can be of varied nature, form and origin. It can be an RNA or, more preferably a DNA. The method can be used to analyse an isolated nucleic acid, or in order to test a composition or a complex sample including a plurality of nucleic acids which are not characterised individually. The presence of a hybridisation can be demonstrated in different ways. In general, the test nucleic acid is labelled before, and the formation of a hybrid is detected by demonstration of the label on the biochip. The labelling can be radioactive, fluorescent, enzymatic, luminescent, chemical, etc. Other detection techniques use visualisation probes, electrical detectors, etc.

Another aspect of the invention is a method for identifying genes associated with a given character trait, including (i) identifying fragments of nucleic acids which are identical between two samples originating from subjects with a common character trait, and (ii) hybridising the fragments identified in this way on a biochip as defined above. Detection of a hybridisation signal makes it possible to locate the fragment(s) on the human genome, and thus to identify one or more genes present in the same, associated with said character trait. The character trait can be a disease (e.g., monogenic or complex genetic disease), a given phenotype (e.g., response to a treatment), etc. Step (i) can be implemented by different techniques, such as those described in application WO00/53802 or in patent U.S. Pat. No. 5,376,526.

Other aspects and advantages of this invention will become clear from reading the following examples, which should be considered as illustrative and not limiting.

### LEGENDS TO THE FIGURES

FIG. 1: Synthetic representation of the covering of the human genome (Build 33) by the 2263 positioned clones.

FIG. 2: Representation curve for determining the threshold value.

FIG. 3: IBD prediction analysis

FIG. 4: Genetic analysis (A) Analysis on the whole of the genome. The signals corresponding to the autosomes give as expected a comparable signal for the 2 individuals. The Cy5 male signal appears clearly reduced for the X chromosome in adequation with the difference of a factor 2 between male and female. (B) Deletion of the dystrophin gene on the X chromosome.

### EXAMPLES

### Example 1

### Selection of the Clones

For selection of the clones, the initial data used were as follows:
collections of clones (Clone Registry @ NCBI)
plaques containing clones (supplier)
mapping of clones on the genome (Golden Path, NCBI)
characteristics of the clones (STS, FISH, ACN, phase.—Golden Path, NCBI).

All of these data have been deposited in a local relational data base.

An XML format file was generated so as to serve as an access to the Clonetrek programme, which synthetically describes all of the properties of the BAC clones taken into account for the selection.

The initial selection was made from available FISH clones (i.e. clones positioned by in-situ hybridisation). We thus had a total of 76 plaques:
VGC: 47 plaques (3294 clones, 2708 positioned)
CSMC: 15 plaques (860 clones, 735 positioned)
CCAP: 14 plaques (719 clones, 662 positioned) on the Golden Path (GP) draft of Dec. 12, 2001.

Because some of these plaques were contaminated, they were eliminated. In total, 41 plaques were used in this study, representing 2460 clones.

Following tagging by Clonetrek (—in maximum distance 900000 and minimal distance 200000 parameters) the algorithm retained 2041 clones with an average spacing of 1.8 Mb. We filled the spaces with additional ‘non-FISH’ clones, either controlled individually by the Research Genetics company, or selected from libraries.

We thus made a selection from a set of approximately 292000 clones (90000 of which positioned on the GP of 28/06/2002) plus a so-called ONCOBAC library (6 plaques i.e. 579 clones of which 108 positioned). We repeated the previous step including the whole ONCOBAC library and the RP11 clones placed on the GP.

After tagging, we obtained 2264 clones (with an average spacing (“gap”) of 1.2 Mb), to which we added the oncobacs which were non-positioned but used on the chip. After re-arrangement of these plaques (“cherry-picking”) the identity of these BACs was verified by terminal sequencing (439 non-sequenced/2779 clones):

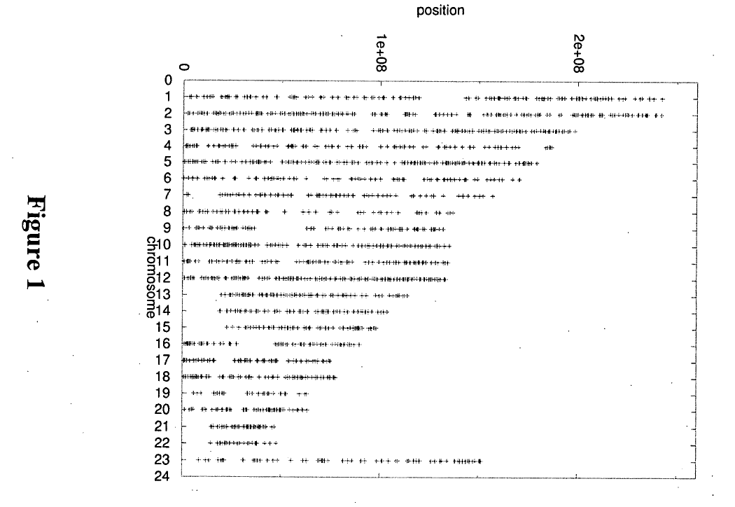

Each pair of sequences was aligned on the sequence from the human genome draft so as to confirm or position the corresponding clone. We used Blast in the up-to-date version on the day of the calculation. Thus, on Build 30 of 28/06/2002 we could confirm 1787 clones, on build 33 of April 2003, 2263 clones were positioned (FIG. 1). The longest gaps are the centromeres of chromosomes 1, 2, 3,4, 5, 6, 7, 10, 11, 12, 16, 17, 18, 19, 20 and 23. Chromosomes 13, 14, 15, 21 and 22 are acrocentromeric, and this explains the absence of clones at their start.

### Example 2

### Producing a Chip

2a. Preparation of the BAC Matrix

After identifying the BACs, each clone was isolated and a mini-preparation of several μg of DNA was obtained by classic methods described in the literature (e.g. the use of kits developed by Qiagen).

An aliquot of about 100 ng of DNA extracted in this way was then amplified. Numerous amplification methods such as Rolling Circle amplification (developed by Amersham) or else DOP-PCR (Degenerate Oligonucleotide Primed-PCR) can be used with enzymes such as templi Phi 29 or the taq polymerase. These amplified DNAs are then purified, for example by precipitation with ethanol or QIAGEN.

The final products are complemented with the components of a solution adapted to printing on slides. These solutions can be 3×SSC or 50% DMSO.

2b-Slide Printing

Numerous types of slide exist on the market. In this example, we opted for slides covered with a layer of amino-silane, distributed by the company Corning (slides called GAPSII).

As with the slides, numerous spotting machines exist. In this study, we implemented the spotting on a Microgrid II produced by the company Biorobotics. The DNA, re-suspended in DMSO solution, were spotted using hollow needles with a reservoir (microspot pins 2500, distributed by Biorobotics), 100 μm in diameter.

The spotting conditions for the whole of our BAC collection which represented 2600 BACs were as follows:
Temperature 20° C., Humidity 50%
Each BAC is spotted in quadruplicate over 2 zones of the slide (1 duplicate per zone)
The pre-spot number is 20 with a variance of 400 μm between each pre-spot.
The variance between each spot is 275 μm.
Re-loading of the matrix every 140 deposits (pre-spot or spot)

After spotting the slides, the DNA deposited on the slides is fixed by UV treatment. Typically this is implemented in a cross linker, exposing the slides to Ultra Violet energy of 70 mJ.

### Example 3

### Hybridisation

Each slide is then subjected to a so-called pre-fixation step, the slides are treated by one of the numerous existing methods which can fix DNA non-specifically.

It can include chemical blocking of the amine groups or else incubation with bovine serum albumin and sodium docecylsulphate. In this study, we used the latter method.

For each slide, the Cy3 and Cy5 labelled DNA are mixed in a buffer solution containing formamide and other components so as to reduce non-specific hybridisation (tRNA, DNA of salmon sperm, Cot1 subscript 0, etc.). After incubation at 70° C., the probes are left for min. 1 hr at 37° C. The probe prepared in this way is then placed in contact on the slide containing the spotted BACs, then the plate is covered with a cover slip. This arrangement is then transferred to a Corning type hybridisation chamber, where several μL of H2O are deposited in each of the two wells present at the ends, making it possible to maintain a certain humidity rate here. After hermetically closing the chamber, this is immersed in a bain marie at 42° C.

Hybridisation times vary between 16 hours and 3 days depending upon the type of hybridised probes. “Hybridising machine” type systems exist which it is also possible to use in this context.

After incubation, the hybridised slides are washed in classic washing solutions so as to eliminate the non-hybridised probes and various a-specific hybridisations.

### Example 4

### Reading the Plates

Numerous fluorescence readers exist for biochips. We opted for a device distributed by Agilent which offers the advantage of having a carrousel making it possible to read 48 slides one after the other.

In this case, we worked with the following settings:
Surface 60 ×21.6 mm
Resolution 10 μm/pixel
Photo-multiplier coefficient (PMT)—100% for the 2 channels.

The images are captured per batch in the device's carrousel (maximum 40). Each image can be re-orientated (flip & rotation) and each wave length stored in a separate file such as to ensure compatibility with the processing software used down the line.

Processing of the image consists of transforming raw data in the form of images into qualitative and quantitative data for each spot. Associated with each type of chip is an information file which indicates its topology and for each point its identity. This identity subsequently makes it possible to link the results to a data base and, for each clone, to obtain its location on the genome.

The analysis of images is made up of a first segmentation step (identification of blocks and spots) and of a second quantification step which calculates a set of numerical data for each spot (co-ordinates, surface, intensity and background noise for each wavelength, ratios, etc.).

According to the Geneprix Pro (version 4.1 ) or Recife context, we use a programme developed internally for collecting this information.

### Example 5

### Examples of Results Obtained

5a. Application to the Identification of Identical Fragments by Descent (“IBD”)

With the aim of validating our chips, we carried out experiments on pairs of individuals with known status.

a. Initial Data:
20 times the pair:CEPH family (Centre of Studies of Human

Polymorphism) 1331 individuals 7 and 9
20 times the pair:CEPH family 1347 individuals 3 and 6
20 times the pair:CEPH family 1362 individuals 3 and 8 full sibs
8 times the pair:CEPH family 1347 individuals 12 and 8
8 times the pair:CEPH family 1347 individuals 13 and 8 Grand-parent—grand-child

The selection of pairs of sibs was made according to the following criteria: [symbol] optimisation of the number of clones, the IBD status of which is known (by genotyping microsatellite markers) [symbol] optimisation of the number of IBD clones for which each of the three status 0, 1, 2 is present.

->Possibility of confronting observed IBD versus known IBD.

b. Analysis of the Data:

10 images per pair of sibs were analysed (total=30), as well as 4 images per grand-parent—grand-child pair. (total 8)

i - analysis of images:
[symbol] reading: The image analyses were implemented using the Genepix programme, version 4.1.
[symbol] Filter: several filters were applied to each image so as to remove any non-analysable or non-informative spots.
   The points which meet the following conditions are preserved:
   o Cy5<40 000 (physical saturation of the spots)
   o Cy3<40 000 (physical saturation of the spots)
   o Cy3-BGCy3 >Average BGCy3
   o Number of replicas per clone >=3
   o variance between the replicas of a same clone <2 * variance (
variance of all of the clones).
   [symbol] (BG=background)
[symbol] Standardisation:
   o Standardisation of the signal Cy3 by signal Cy3 of all of the spots.
   o Standardisation of the signal Cy5 by signal Cy5 of all of the spots.
[symbol] Annotation:
Positioning of the clones on the genome (position in pairs of bases as well as by cytogenetic position obtained by FISH)

A file for output in pairs is produced at the end of the whole computer process. All that is preserved in the file are the names and positions of the clones as well as the median of the ratio of the signals between the replicas:

$$Ratio = \frac{[ {\sum }_{i = 1}^{N} ( Cy {5}_{i} - BGcy {5}_{i} ) ] / N}{[ {\sum }_{i = 1}^{N} ( Cy {3}_{i} - BGcy {3}_{i} ) ] / N}$$

N=number of pixels per spot.

ii IBD Prediction:

a - ) Sliding Average:

For each clone an average of the ratios is calculated taking into account the neighbouring clones on each independent chromosome: The streamlined ratio makes it possible for us to determine the threshold value for which we can distinguish the IBD of the non-IBD.

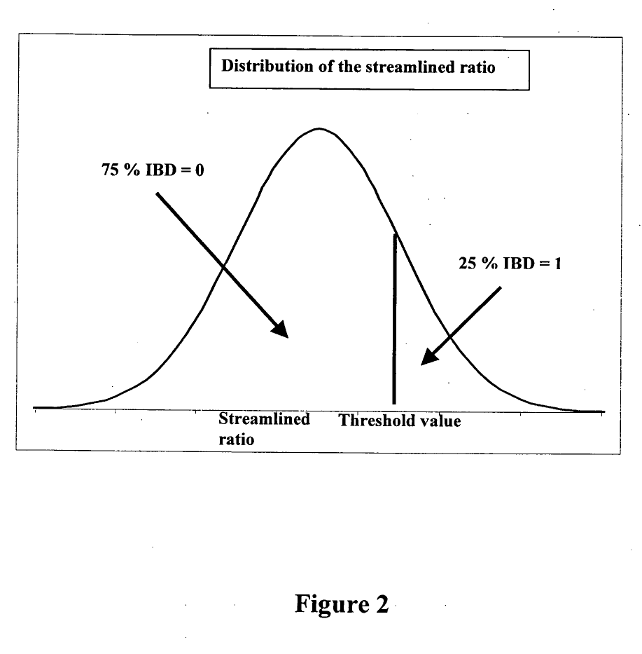

b - ) Determining the Threshold Value (FIG. 2).

In order to determine the threshold value for which one will be able to distinguish the IBD status of the non-IBD, two factors are used:
[symbol] The Gaussian distribution of the ratio
[symbol] the probability between two sibs of being IBD=0.75
   so the threshold is the value for which one has 75% of clones on one side and 25% of clones on the other (FIG. 2).

c -) IBD Prediction:

Each value of the streamlined ratio is then compared to the threshold value:
[symbol] Streamlined ratio>Threshold: IBD=1
[symbol] Streamlined ratio<=Threshold: IBD=0

A filter is then applied to a motif search:
[symbol] IBD clone on its own in the middle of the non-IBD region: motif: 0 0 1 0 0 recoding to 0 0 0 0 0
[symbol] non-IBD clone on its own in the IBD region motif: 1 1 0 1 1 re-coding to 1 1 1 1

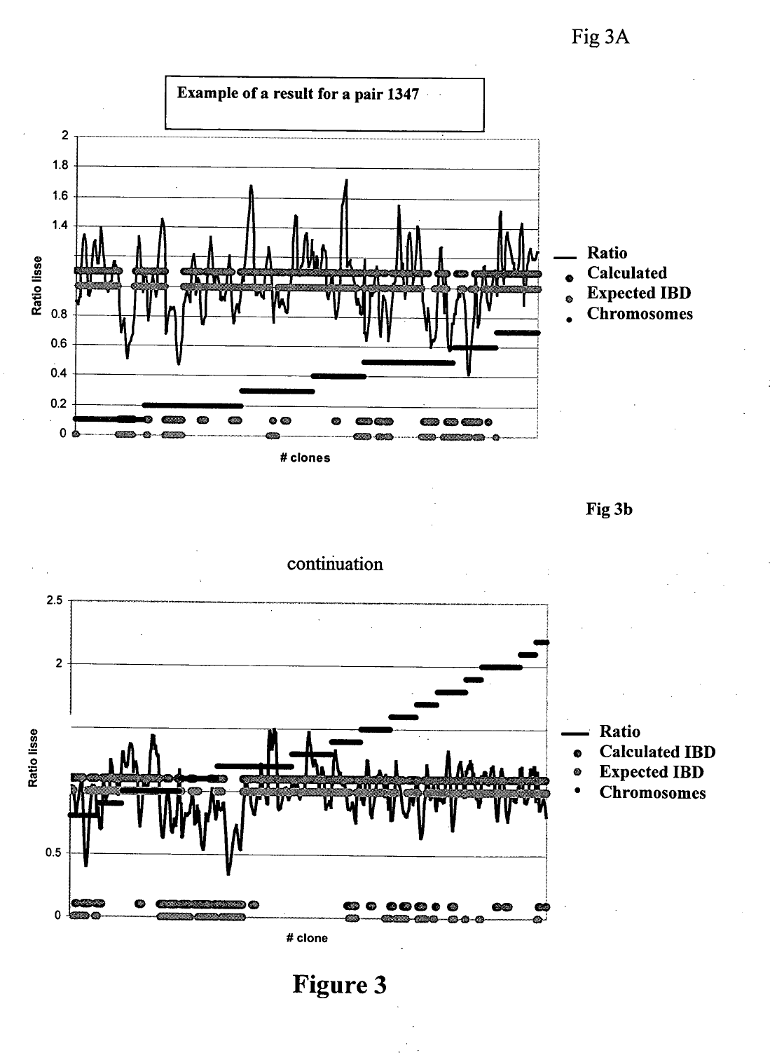

iii Result (FIG. 3):

The IBD prediction analysis was implemented on all of the available CEPH pairs. The results obtained are shown in FIG. 3 and illustrate the reliability of the chips of the invention. These results have been compared to the IBD status expected for the clones for which one has IBD information: Three percentages were calculated:
[symbol] observed IBD=expected IBD (% success)
[symbol] observed IBD≠expected IBD:
   observed=0 expected=1: false negative [symbol] observed=1 expected=0: false positive

5b. Application to the detection of a difference in the number of copies of a fragment of the genome including deletion of a gene involved in an illness.

With a sample which includes a deletion which covers 0.2% of the genome, the IntegraGen platform has made it possible to detect a portion of the deletion which only represents a 1/20,000th of the genome.

A female human DNA taken from a normal individual was marked with Cy3 and a male human DNA taken from an individual suffering from Duchesne myopathy with Cy5. This ailment is accompanied by a deletion of between 5 and 10 Mb in the 5′ part of the dystrophin gene and the adjacent regions.

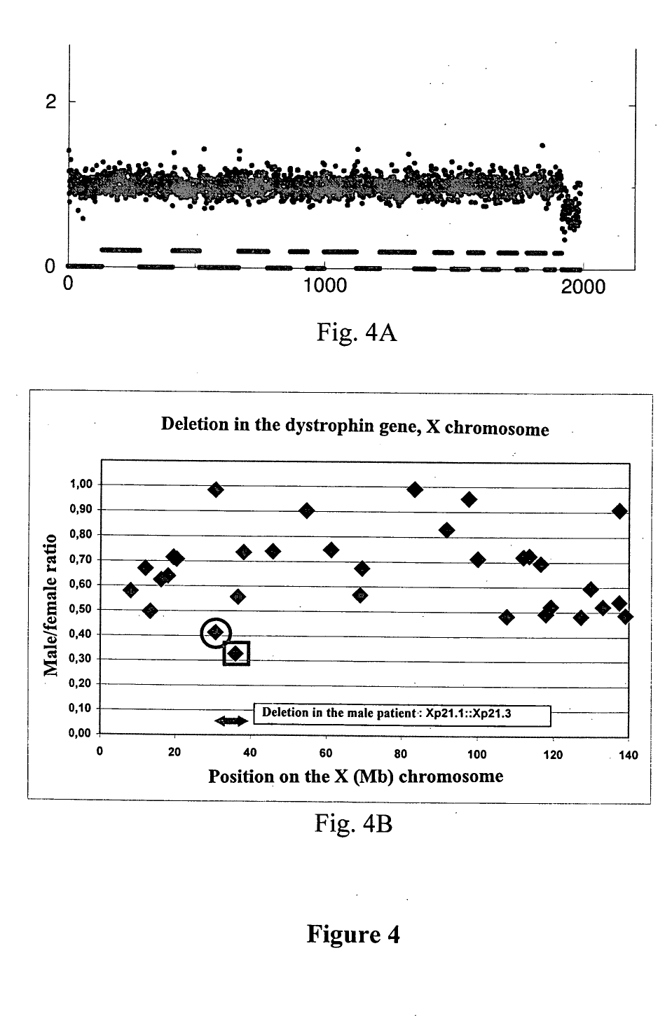

The patient is also suffering from chronic granulomatosis (CGD), pigmentary retinitis and mental handicap (Coriell Collection GM07947). A quantity of DNA comparable to that which it is possible to obtain by biopsy was generically amplified, and hybridised to a chip as described in example 2. The results obtained are shown in FIG. 4.

They show that a region of more than 5 Mb was demonstrated by cytogenetic analyses in bands Xp21.3 to Xp21.1. The clone represented by a square is located in the deletion. The clone represented by a circle is close to the cytogenetic limit, its relative signal is clearly between the X chromosome cluster values observed during the male/female hybridisations and the deletion zone. For this reason, it can be concluded that it contains the “breakpoint” for the deletion.

In view of these observations, it can be affirmed that a chip according to the invention including a contig of covering BACs in this region would make it possible to determine more precisely breakage points in the deletion and to have more advanced knowledge of the neighbouring genes of the dystrophin playing a role in the appearance of complex phenotypical traits.

1-21. (canceled)

22. A method for producing a biochip including a support on which is immobilized a set of polynucleotides, which method comprises the steps of:
(i) obtaining BAC clones including a nucleic insert corresponding to or specific to a portion of a human genome,
(ii) selecting, from the BAC clones thus obtained, a set of BAC clones including a single nucleic insert in the human genome, the BAC clones of the selected set including nucleic inserts substantially uniformly distributed over the human genome, the selection step comprising:
(a) eliminating non-single clones;
(b) eliminating clones sharing a same STS;
(c) eliminating STS which are marked at least two different places on the human genome;
(d) classifying clones as a function of their position on the genome to defining neighbouring clones; and
(e) eliminating clones by applying an iterative method so as to obtain in particular substantially uniform distribution of the finally selected clones over the human genome; and
(iii) depositing, on a support, the selected clones or the nucleic inserts contained in said clones, or part of them, under conditions enabling the deposited clones or inserts to hybridise with a nucleic acid having a complementary sequence.

23. The method according to claim 22, wherein step (i) comprises obtaining a collection of BAC clones including nucleic inserts likely to cover the whole sequence of the human genome.

24. The method according to claim 22, wherein the BAC clones of the selected set include nucleic inserts spaced apart from one another by an interval of about 1 Mb.

25. The method according to claim 22, wherein selection steps a) to e), or part of them, are repeated at least once until a set of BAC clones is obtained including a single insert in the human genome, the BAC clones of the selected set furthermore including nucleic inserts substantially uniformly distributed over the human genome, and covering the whole human genome.

26. The method according to claim 22, wherein step a) comprises compilation or analysis of the information known for a clone by computer analysis of the sequence of the nucleic insert contained therein and/or by biological experiments.

27. The method according to claim 22, wherein step e) comprises a first sub-step (e₁) of extracting a sub-set of BAC clones likely to be eliminated from the finally selected set of clones, and a second sub-step (e₂), during which a second criterion is applied to the elements of the sub-set of clones likely to be eliminated, so as to determine the single clone of this sub-set which will be effectively eliminated.

28. The method according to claim 27, wherein the first sub-step (e₁) of extracting from the sub-set BAC clones likely to be eliminated comprises applying a first rejection criterion that comprises defining a maximum authorized distance between two neighbouring clones of the finally selected set of clones.

29. The method according to claim 27, wherein the second criterion applied during the second sub-step (e2) includes requiring a minimum distance between two neighbouring clones of the finally selected set of clones.

30. The method according to claim 28, wherein the first criterion is based upon a maximum distance of 1.5 Mb and the second criterion requires a minimum distance of 0.7 Mb.

31. The method according to claim 22, wherein step (ii) of selecting BAC clones is implemented by means of a computer programme.

32. The method according to claim 22, wherein, in step (iii), the BAC clones or the nucleic inserts that they contain, or part of these clones or inserts, are deposited on a support by direct coupling on the support by an interaction with a complementary oligonucleotide, or by means of a spacer arm.

33. The method according to claim 32, wherein the support is level and/or rigid.

34. The method according to claim 32, wherein the support is made with a base of materials chosen from glass, plastic, polymer, metal, biological materials, silicons and nylon.

35. The method according to claim 34, wherein the support is a glass slide.

36. The method according to claim 22, wherein, during step (iii), depositing is implemented according to a pre-specified arrangement and/or density.

37. The method according to claim 22, wherein, prior to step (iii), the clones of the selected set are sub-cultured, amplified, characterised and/or stored.

38. The method for producing a biochip including a support on which has been immobilized a set of polynucleotides, which method comprising the steps of:
(i) obtaining BAC clones including a nucleic insert corresponding to or specific to a portion of a human genome;
(ii) selecting, from the BAC clones thus obtained, a set of BAC clones including a single insert in the human genome, BAC clones of the selected set including nucleic inserts substantially uniformly distributed over the human genome, the selection step comprising:
(a) eliminating non-single clones;
(b) eliminating clones sharing a same STS;
(c) eliminating STS which are marked at at least two different places on the human genome;
(d) classifying clones as a function of their position on the genome to defining neighbouring clones; and
(e) eliminating clones by applying an iterative method so as to obtain in particular substantially uniform distribution of the finally selected clones over the human genome;
(iii) amplifying the BAC clones of the selected set and/or the nucleic inserts that they contain; and
(iv) depositing, on a support, the selected clones or the nucleic inserts contained in said clones, or part of them, under conditions enabling deposited clones or inserts to hybridise with a nucleic acid having a complementary sequence.

39. A biochip that comprises a support on which is immobilized a set of BAC clones including a nucleic insert corresponding to or specific to a portion of a human genome, each clone including a single insert in the human genome and carrying an STS not shared by any other insert of the BAC clones of the set, the BAC clones of the set including nucleic inserts substantially uniformly distributed over the human genome.

40. A method for identifying or locating a nucleic acid on the human genome, comprising placing a nucleic acid in contact with a biochip, in conditions enabling hybridisation between complementary sequences, detection of a hybridisation signal, and identification of the position of the nucleic acid on the genome by identification of the clones involved in the hybridisations, wherein the biochip comprises a support on which is immobilized a set of BAC clones including a nucleic insert corresponding to or specific to a portion of a human genome, each clone including a single insert in the human genome and carrying an STS not shared by any other insert of the BAC clones of the set, the BAC clones of the set including nucleic inserts substantially uniformly distributed over the human genome.

41. A method for identifying genes associated with a given character trait, comprising (i) identifying fragments of nucleic acids identical between at least two samples taken from subjects having a common character trait, and (ii) hybridising fragments thus identified on a biochip and, (iii) detecting a hybridisation signal, making it possible to locate the fragment(s) on the human genome and thus to identify one or more genes present therein, associated with said character trait, wherein the biochip comprises a support on which is immobilized a set of BAC clones including a nucleic insert corresponding to or specific to a portion of a human genome, each clone including a single insert in the human genome and carrying an STS not shared by any other insert of the BAC clones of the set, the BAC clones of the set including nucleic inserts substantially uniformly distributed over the human genome.
